(19) United States
(12) Patent Application Publication
(10) Pub. No.: US 2022/0017442 A1
(43) Pub. Date: Jan. 20, 2022

(54) METHOD FOR PRODUCING A HYDROXY COMPOUND BY DECARBOXYLATION IN THE PRESENCE OF A BRONSTED BASE

(71) Applicant: Covestro Intellectual Property GmbH & Co. KG, Leverkusen (DE)

(72) Inventors: Jens Langanke, Mechernich (DE); Erik Sluyts, Brasschaat (BE); Jan Heijl, Lokeren (BE); Niklas Meine, Düsseldorf (DE)

(21) Appl. No.: 17/294,932

(22) PCT Filed: Dec. 2, 2019
(86) PCT No.: PCT/EP2019/083211

§ 371 Date: May 18, 2021

(30) Foreign Application Priority Data
Dec. 7, 2018 (EP) 18211034.6

(51) Int. Cl. C07C 37/56

(52) U.S. Cl. CPC C07C 37/56

(57) ABSTRACT

The invention relates to a method for producing a specific hydroxy compound by decarboxylating a specific carboxylic acid compound or a salt of said carboxylic acid compound in the presence of a Bronsted base. The invention also relates to a method for producing a diaryl carbonate or a bisphenol, to a method for producing a polycarbonate and to a use of a Bronsted base during the reaction of the decarboxylation of a specific carboxylic acid compound or a salt of said carboxylic acid compound.

The present invention relates to a process for producing a specific hydroxy compound by decarboxylation of a specific carboxylic acid compound or of a salt of said carboxylic acid compound in the presence of a Brønsted base, to a process for producing a diaryl carbonate or a bisphenol, to a process for producing a polycarbonate, and to the use of a Brønsted base in the reaction for the decarboxylation of a specific carboxylic acid compound or of a salt of said carboxylic acid compound.

Phenols having different substitution patterns on the aromatic ring are the starting compounds for various monomers and thus also for the polymers resulting therefrom. The production of such phenols from sustainable raw materials is a major challenge. One option for producing biobased phenol is the direct fermentation of sugars, as described for example in WO 2014/076113 A1. However, phenol is toxic to the microorganism described therein and its removal from the aqueous fermentation broth is also laborious. Hydroxybenzoic acids such as 4-hydroxybenzoic acid, 2-hydroxybenzoic acid, and 3-hydroxybenzoic acid can likewise be produced from sugars by fermentation. Since they are generally less toxic to the microorganisms used, higher yields can usually be achieved compared to phenol. Hydroxybenzoic acids can be crystallized and separated from the fermentation broth. A subsequent decarboxylation of 4-hydroxybenzoic acid to phenol has also previously been described. JP 2016-23136 A describes the reaction using a heterogeneous catalyst in water as solvent. A. S. Lisitsyn in Applied Catalysis A: General 332; 2007 (166-170) describes decarboxylation in diphenyl ether using a copper catalyst. L. J. Goossen et al. in Chem Cat Chem 2010, 2, 430-442 describe decarboxylation using a silver or copper catalyst in NMP as solvent. Dalton Transactions (24), 4683-4688; 2009 also describes decarboxylation in toluene.

However, the methods described all have potential for improvement in respect of the reaction conditions, yield, and selectivity.

The object of the present invention therefore was to provide a process for producing specific hydroxy compounds of the formula (I) by decarboxylation of a carboxylic acid compound of the formula (II) or of a corresponding salt of said carboxylic acid compound of the formula (II), thereby improving at least one disadvantage of the prior art. In particular, the object of the present invention was to provide a process that affords the hydroxy compound of the formula (I) in high yield. This high yield should preferably be achieved in a correspondingly short time. The yield should particularly preferably be higher than in the processes described in the prior art. The reaction time at correspondingly high yield should likewise preferably be shorter than in the processes described in the prior art.

At least one, preferably all, of the abovementioned objects were achieved by the present invention. It was surprisingly found that the decarboxylation of a carboxylic acid compound of the formula (II) or of a corresponding salt of said carboxylic acid compound of the formula (II) proceeds particularly effectively when at least one Brønsted base is present during the decarboxylation reaction. The yield of the desired hydroxy compound of the formula (I) is preferably even higher than under the conditions described in the prior art. This is also preferably achieved in a shorter time than under the conditions described in the prior art. In addition, high selectivity was observed. The high selectivity in combination with high yield offers the particular advantage that a reaction product is obtained from which the starting material (a carboxylic acid compound of the formula (II) or corresponding salt of said carboxylic acid compound of the formula (II)) does not need to be laboriously separated, but can instead be used for further reactions.

The invention accordingly provides a process for producing a hydroxy compound of the formula (I)

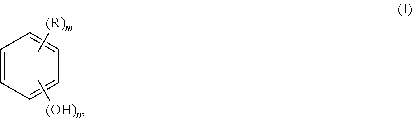

in which

R is a linear or branched alkyl group having 1 to 6 carbon atoms,

n is 1 or 2, and

m is 0, 1, 2, or 3,

by decarboxylation of a carboxylic acid compound of the formula (II) or of a corresponding salt of said carboxylic acid compound of the formula (II)

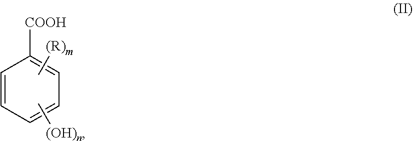

in which R, n, and m are as defined above,

optionally with the use of at least one heterogeneous catalyst,

the process of the invention being characterized in that at least one Brønsted base is present during the decarboxylation reaction.

The term “Brønsted base” is known to those skilled in the art. In particular, they understand it as meaning a compound that can accept a proton and thus acts as a proton acceptor. The process of the invention is preferably characterized in that the at least one Brønsted base is selected from the group consisting of sodium hydroxide, potassium hydroxide, sodium phenolate, sodium acetate, sodium phosphate, and any desired mixtures thereof. The at least one Brønsted base is particularly preferably selected from the group consisting of sodium hydroxide, potassium hydroxide, sodium phenolate, and any desired mixtures thereof. The at least one Brønsted base is very particularly preferably sodium hydroxide. This is particularly advantageous because it makes the hydroxy compound of the formula (I) that is produced particularly suitable for use in the production of a bisphenol, a diaryl carbonate, and/or a polycarbonate, since sodium hydroxide is often also required for the synthesis of these substances. This means that any contamination of the hydroxy compounds of the formula (I) by the Brønsted base does not significantly interfere with their further conversion into the desired target product.

A nitrogen-containing Brønsted base such as ammonia, a primary amine, for example monomethylamine or monoethylamine, or a secondary amine, for example dimethylamine or diethylamine, or a mixture of two or more of these nitrogen-containing Brønsted bases, is disadvantageous for the process of the invention and should therefore be avoided. A Brønsted base of this type is disadvantageous because contamination by a nitrogen-containing Brønsted base of this type makes the hydroxy compound of the formula (I) that is produced unsuitable for use in the production of a bisphenol, a diaryl carbonate, and/or a polycarbonate. The reason for this is that, when the hydroxy compound of the formula (I) is used for the production of polycarbonates, a nitrogen-containing Brønsted base of this kind can cause side reactions that result in the formation of carbamate compounds. Such carbamate compounds are undesirable in polycarbonates because they incorporate nitrogen into the polymer chain, which downgrades the properties of the polycarbonate, as is already known from DE10300598A1. This means that any contamination of the hydroxy compounds of the formula (I) by a Brønsted base of this type can seriously interfere with the further conversion of the hydroxy compounds of the formula (I) into the desired target product.

The process of the invention therefore preferably excludes the use of ammonia, primary amines, and secondary amines as the Brønsted base, and the process of the invention particularly preferably completely excludes the use of ammonia, primary amines, and secondary amines.

The use of nitrogen-containing Brønsted bases that are tertiary amines, for example triethylamine, tributylamine or N-ethylpiperidine, is however not excluded from the process of the invention. The use of tertiary amines does not result in carbamate formation if these tertiary amines should still be present as contaminants in the hydroxy compound of the formula (I) that is produced.

The inventors observed experimentally that the addition of a Brønsted base in the decarboxylation reaction resulted in a higher yield of the desired target product than was obtained under otherwise identical reaction conditions but without a base. In addition, it could be seen from the premature termination of the reaction that the yield obtained by adding the Brønsted base at a significantly earlier point in time was the same as the yield obtained without addition of the Brønsted base. These observations were particularly surprising since the decarboxylation is in the literature often catalyzed by acid. It is preferable here when the at least one Brønsted base is present in a concentration of 0.0001 mol/L to 20 mol/L, particularly preferably 0.001 mol/L to 5 mol/L, particularly preferably 0.001 mol/L to 1 mol/L, particularly preferably 0.01 mol/L to 0.1 mol/L, particularly preferably 0.001 mol/L to 0.05 mol/L, and very particularly preferably 0.009 mol/L to 0.02 mol/L, based on the entire decarboxylation reaction mixture. At these concentrations, it has been found that the Brønsted base shows the desired catalytic activity in the decarboxylation reaction and at the same time the concentration of the Brønsted base is sufficiently low that adverse effects such as corrosion of the devices used for the reaction can be kept to a minimum.

It has been found to be advantageous that the process of the invention is carried out in solution. The terms “dissolve” and “in solution” are in accordance with the invention to be understood as having the meanings known to those skilled in the art. The terms “dissolve” and “in solution” preferably mean that, when filtering a liquid in which a substance is dissolved, no solid can be separated off using customary filter methods. In accordance with the invention, it is therefore preferable that the carboxylic acid compound of the formula (II) or corresponding salt of the carboxylic acid compound of the formula (II) is dissolved in a solvent before carrying out the decarboxylation reaction. The dissolution process can also take place with heating in a manner known to those skilled in the art. The term “solvent” is known to those skilled in the art. However, it has been found to be advantageous in accordance with the invention that the decarboxylation reaction is carried out in an aqueous medium. This preferably means that at least part of the solvent used is water. The term “aqueous medium” is particularly preferably understood as meaning that at least 10% by volume of the solvent used is water, preferably at least 50% by volume, particularly preferably at least 80% by volume, and further preferably at least 90% by volume. The aqueous medium very particularly preferably consists of water. A customary water quality in which appropriate ions are present can however be assumed here.

This embodiment offers the particular advantage that organic solvents are not used or are used only in small amounts. This means they do not then have to be removed in an additional step. In particular, not using an organic solvent means that no other undesired organic contaminants are introduced into the system.

As already described above, the addition of the at least one Brønsted base allowed the reaction time to be shortened by comparison with an otherwise identical reaction without base. It is therefore preferable in accordance with the invention that the reaction time of the decarboxylation reaction is longer than 5 min and shorter than 48 h, particularly preferably longer than 25 min and shorter than 24 h, likewise preferably longer than 40 min and shorter than 12 h, and very particularly preferably longer than 1 h and shorter than 5 h. Those skilled in the art know that, at relatively high concentrations of the catalyst, in this case of the Brønsted base, shorter reaction times may be sufficient for a high yield.

The process of the invention can be a batch process, semi-batch process or continuous process.

The process of the invention is preferably executed at a temperature of 20 to 400° C., more preferably at 100 to 350° C., particularly preferably at 150 to 300° C., and most particularly preferably from 160 to 250° C.

The process of the invention is preferably used for producing a hydroxy compound of the formula (I) shown above, in which R is a tert-butyl, propyl or methyl group, n is 1 or 2, preferably 1, and m is 0, 1, 2 or 3. The process of the invention is particularly preferably used to produce 4-propylphenol, ortho-, para- or meta-methylphenol (cresols), 2,4-dimethylphenol, 2,5-dimethylphenol, 4-tert-butylphenol or phenol. The process of the invention is very particularly preferably characterized in that the hydroxy compound of the formula (I) is phenol.

In accordance with the invention, the carboxylic acid compound of the formula (II) or salt of the carboxylic acid compound of the formula (II) are occasionally also collectively referred to as the carboxylic acid compound of the formula (II). However, unless otherwise stated, this always means the free acid and/or the salt. According to the invention, it is also possible to use mixtures of different carboxylic acid compounds of the formula (II) or of different salts of the carboxylic acid compounds of the formula (II) or else mixtures of at least one carboxylic acid compound of the formula (II) with at least one salt of the carboxylic acid compound of the formula (II).

In the process of the invention it is preferable that the cation of the salt of the carboxylic acid compound of the formula (II) is selected from the group consisting of alkali metal cations, alkaline earth metal cations, ammonium, phosphonium, cations of manganese, iron, cobalt, nickel, copper, zinc, molybdenum, cadmium, and any desired mixtures thereof. The cation of the salt of the carboxylic acid compound of the formula (II) is particularly preferably selected from the group consisting of alkali metal cations, alkaline earth metal cations, and mixtures thereof.

In addition, it is preferable in accordance with the invention that the carboxylic acid compound of the formula (II) or corresponding salt of the carboxylic acid compound of the formula (II) is selected from the group consisting of 2-hydroxybenzoic acid, 4-hydroxybenzoic acid, and the corresponding salts. Very particularly preference is given to 4-hydroxybenzoic acid or the corresponding salt.

When a catalyst is used in the process of the invention, all heterogeneous catalysts that are active in a decarboxylation reaction are in principle suitable. These are known to those skilled in the art. The heterogeneous catalyst used in the process of the invention is preferably selected from the group consisting of Al₂O₃, H₃PO₄ supported on Al₂O₃, PtClₓ supported on Al₂O₃, Cu/Al/Ga-MOFs, Pt—Al-MOFs, palladium supported on activated carbon, platinum supported on activated carbon, zeolites such as ZSM-5, HZSM-5, Fe₂O₃ supported on MCM-41 (Mobil Composition of Matter No. 41), Fe₂O₃ supported on Al-MCM-41, Pt supported on SAPO-34 (silicoaluminophosphate), Pt supported on SAPO-11, Pt hydrotalcite, Pt supported on SiO₂, and any desired mixtures thereof. The process of the invention is particularly preferably characterized in that the at least one heterogeneous catalyst optionally used is a zeolite. The process of the invention is very particularly preferably characterized in that the zeolite has a faujasite structure.

Zeolites and in particular zeolites having a faujasite structure are known to those skilled in the art. The crystal structure of faujasite is identical to that of the synthetic zeolite Y. The basic element of the faujasite framework is sodalite cages, which are connected to one another via hexagonal prisms. Very particularly preference is given to the zeolite type Y catalyst used according to the invention.

According to the invention, the process can be carried out with and without a catalyst. It is preferable that the process is carried out either entirely without a catalyst or with one of the preferred catalysts mentioned above. In a reaction described as “without a catalyst”, the presence of the at least one Brønsted base is not excluded, but required according to the invention.

In one aspect of the invention, it is further preferable that the carboxylic acid compound of the formula (II) or corresponding salt of the carboxylic acid compound of the formula (II) was obtained by fermentation or from sugars, lignocellulose, lignocellulose-containing materials, furans, and/or lignin. The carboxylic acid compound of the formula (II) or corresponding salt of the carboxylic acid compound of the formula (II) is thus preferably biobased. For the purposes of the present invention, the expression “biobased” is understood as meaning that the relevant chemical compound is at the filing date available and/or obtainable via a renewable and/or sustainable raw material and/or preferably is such a renewable and/or sustainable raw material. A renewable and/or sustainable raw material is preferably understood as meaning a raw material that is regenerated by natural processes at a rate that is comparable to its rate of depletion (see CEN/TS 16295:2012). The expression is used in particular to differentiate it from raw materials produced from fossil raw materials, also referred to in accordance with the invention as petroleum-based. Whether a raw material is biobased or petroleum-based can be determined by the measurement of carbon isotopes in the raw material, since the relative amounts of the carbon isotope C14 are lower in fossil raw materials. This can be done, for example, in accordance with ASTM D6866-18 (2018) or ISO16620-1 to -5 (2015) or DIN SPEC 91236 2011-07.

In accordance with the invention, the term “petroleum-based” is preferably used to describe those compounds that have a C14 isotope content of less than 0.3×10⁻¹², particularly preferably of 0.2×10⁻¹², and very particularly preferably of 0.1×10¹².

Those skilled in the art know how to obtain the carboxylic acid compound of the formula (II) or corresponding salt of the carboxylic acid compound of the formula (II) by fermentation or from sugars, lignocellulose, lignocellulose-containing materials, furans, and/or lignin. This is described for example in WO 2015174446, WO 2015156271, US20040143867, Appl. Environ Microbiol 84 2018:e02587-17, WO2016114668, Biomass and Bioenergy 93:209-216 October 2016, Biotechnol Bioeng. 2016 July; 113(7):1493-503, ACS Catal., 2016, 6 (9), pp. 6141-6145 or Biotechnol. Bioeng., 113: 1493-1503, Appl Microbiol Biotechnol. 2018 October; 102(20):8685-8705, Microbiology. 1994 April; 140 (Pt 4):897-904, Journal of Biotechnology 132 (2007) 49-56, WO2000018942, U.S. Pat. No. 6,030,819, EP2698435, Bioprocess Biosyst Eng (2017) 40: 1283, U.S. Pat. Nos. 2,996,540, 9,206,449, Nature 2014, 515, 249-252, Biomass and Bioenergy 93 (2016) 209-216, 3 Biotech. 2015 October; 5(5): 647-651, Appl Environ Microbiol. 2018 Mar. 15; 84(6): e02587-17, U.S. Pat. No. 3,360,553A.

In this aspect of the invention, it is particularly advantageous that the use of a biobased carboxylic acid compound of the formula (II) or of a corresponding salt of the carboxylic acid compound of the formula (II) affords a biobased hydroxy compound of the formula (I). This can in turn be used to produce further biobased compounds, for example diaryl carbonates, bisphenols or polycarbonates, ultimately providing access to biobased polymers and allowing them to be produced in an efficient and cost-effective way.

A further aspect of the present invention provides a process for producing a diaryl carbonate or a bisphenol, which is characterized in that it comprises the following steps:
(i) executing the process in all configurations and preferences so as to obtain a hydroxy compound of the formula (I) and
(ii) reacting the hydroxy compound of the formula (I) from step (i) with at least one ketone to obtain a bisphenol or reacting the hydroxy compound of the formula (I) from step (i) with phosgene to obtain a diaryl carbonate.

It has been found to be particularly advantageous that the high selectivity in process step (i), as already described above, affords the hydroxy compound of the formula (I) in particularly high purity. This can then be processed further directly without laborious workup. Any separation of the heterogeneous catalyst, if present, and/or of any solvent present is preferably carried out beforehand. The separation of any catalyst present is known to those skilled in the art. It can be achieved for example by filtration. Those skilled in the art also know how to remove any solvent present. This can be effected for example by distillation, absorption or similar separation processes. Depending on the concentration of the Brønsted base used, this can optionally also be neutralized first or else remain in the system. Neutralization can be effected for example by adding HCl. The NaCl formed can prior to further use either be separated by a customary method known to those skilled in the art or can, in a similar manner to the Brønsted base, remain in the mixture, since low concentrations thereof have negligible effect on the course of the reaction in the process of the invention for the production of a diaryl carbonate or a bisphenol.

According to the invention, it is in this situation particularly preferable that the carboxylic acid compound of the formula (II) or salt of the carboxylic acid compound of the formula (II) is 4-hydroxybenzoic acid or the corresponding salt.

Processes for producing diaryl carbonates or bisphenols are known to those skilled in the art. Diaryl carbonates can be produced for example by reacting the hydroxy compound of the formula (I) with phosgene in a known manner Bisphenols can be obtained by reacting the hydroxy compound of the formula (I) with a ketone in a known manner. In these processes, the other reactants, such as the ketones, can likewise be biobased or petroleum-based, preferably biobased. This allows products with different proportions of biobased carbon to be selectively obtained.

There are currently different labels according to the point from which a product may be described as “biobased” (see inter alia the certification program for “biobased” products according to ASTM D6866-18 (2018) or ISO16620-1 to -5 (2015) or DIN SPEC 91236 2011-07 from TÜV Rheinland®). The requirement for these different labels is a certain percentage of biobased carbon in the product. The process of the invention makes it possible to easily adjust the proportion of biobased carbon.

Preferred bisphenols that can be prepared with the process of the invention are those of the formula (2a)

HO—Z—OH (2a),

in which

Z is an aromatic radical having 6 to 30 carbon atoms that may contain one or more aromatic rings, may be substituted, and may contain aliphatic or cycloaliphatic radicals or alkylaryls or heteroatoms as bridging elements.

Z in formula (2a) is preferably a radical of the formula (3)

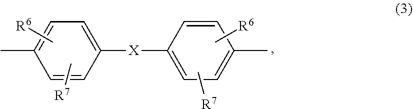

in which

R⁶ and R⁷ are independently H, C₁ to C₁₈ alkyl, C₁ to C₁₈ alkoxy, halogen such as Cl or Br or are each optionally substituted aryl or aralkyl, preferably H or C₁ to C₁₂ alkyl, particularly preferably H or C₁ to C₈ alkyl, and very particularly preferably H or methyl, and

X is a single bond, —SO₂—, —CO—, —O—, —S—, C₁ to C₆ alkylene, C₂ to C₅ alkylidene or C₅ to C₆ cycloalkylidene, which may be substituted by C₁ to C₆ alkyl, preferably methyl or ethyl, or else C₆ to C₁₂ arylene, which may optionally be fused with further heteroatom-containing aromatic rings.

X is preferably a single bond, C₁ to C₅ alkylene, C₂ to C₅ alkylidene, C₅ to C₆ cycloalkylidene, —O—, —SO—, —CO—, —S—, —SO₂—

or is a radical of the formula (3a)

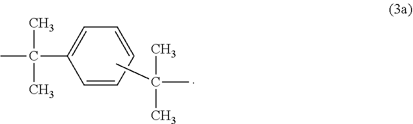

Examples of bisphenols are: dihydroxybenzenes, dihydroxydiphenyls, bis(hydroxyphenyl)alkanes, bis(hydroxyphenyl)cycloalkanes, bis(hydroxyphenyl)aryls, bis(hydroxyphenyl) ethers, bis(hydroxyphenyl) ketones, bis(hydroxyphenyl) sulfides, bis(hydroxyphenyl) sulfones, bis(hydroxyphenyl) sulfoxides, 1,1′-bis(hydroxyphenyl)diisopropylbenzenes, and the ring-alkylated and ring-halogenated compounds thereof.

Preferred bisphenols are 4,4′-dihydroxydiphenyl, 2,2-bis(4-hydroxyphenyl)-1-phenylpropane, 1,1-bis(4-hydroxyphenyl)phenylethane, 2,2-bis(4-hydroxyphenyl)propane, 2,4-bis(4-hydroxyphenyl)-2-methylbutane, 1,3-bis[2-(4-hydroxyphenyl)-2-propyl]benzene (bisphenol M), 2,2-bis(3-methyl-4-hydroxyphenyl)propane, bis(3,5-dimethyl-4-hydroxyphenyl)methane, 2,2-bis(3,5-dimethyl-4-hydroxyphenyl)propane, bis(3,5-dimethyl-4-hydroxyphenyl) sulfone, 2,4-bis(3,5-dimethyl-4-hydroxyphenyl)-2-methylbutane, 1,3-bis[2-(3,5-dimethyl-4-hydroxyphenyl)-2-propyl]benzene, and 1,1-bis(4-hydroxyphenyl)-3,3,5-trimethylcyclohexane (bisphenol TMC).

Particularly preferred bisphenols are 4,4′-dihydroxydiphenyl, 1,1-bis(4-hydroxyphenyl)phenylethane, 2,2-bis(4-hydroxyphenyl)propane, 2,2-bis(3,5-dimethyl-4-hydroxyphenyl)propane, 1,1-bis(4-hydroxyphenyl)cyclohexane and 1,1-bis(4-hydroxyphenyl)-3,3,5-trimethylcyclohexane (bisphenol TMC).

Preferred diaryl carbonates that can be prepared with the process of the invention are those of the formula (2)

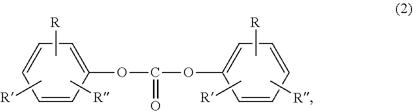

where R, R′ and R″ may each independently be the same or different and are hydrogen, optionally branched C1-C34 alkyl, C7-C34 alkylaryl or C6-C34 aryl; in addition R can also denote —COO—R′″, where R′″ is optionally branched C1-C34 alkyl, C7-C34 alkylaryl or C6-C34 aryl. Such diaryl carbonates are described for example in EP-A 1 609 818. Preference is given to diphenyl carbonate, 4-tert-butylphenyl phenyl carbonate, di(4-tert-butylphenyl) carbonate, biphenyl-4-yl phenyl carbonate, di(biphenyl-4-yl) carbonate, 4-(1-methyl-1-phenylethyl)phenyl phenyl carbonate, and di[4-(1-methyl-1-phenylethyl)phenyl] carbonate. Very particular preference is given to substituted or unsubstituted, preferably unsubstituted, diphenyl carbonate.

A further aspect of the present invention provides a process for producing a polycarbonate through the polymerization of at least one bisphenol and/or of at least one diaryl carbonate, characterized in that either
(A) the at least one bisphenol is produced by the above-described process of the invention for the production of a bisphenol in all configurations and preferences and/or the at least one diaryl carbonate was produced by the above-described process of the invention for the production of a diaryl carbonate in all configurations and preferences or
(B) that a hydroxy compound of the formula (I) is produced by the process described above for the production of a hydroxy compound of the formula (I) in all configurations and preferences and is then used as a chain terminator in the polymerization of the polycarbonate by the interfacial process.

Such processes for producing polycarbonate by polymerization of diaryl carbonates and/or bisphenols are known to those skilled in the art. For example, the bisphenols and any branching agents can be dissolved in an aqueous alkaline solution and reacted with a carbonate source such as phosgene optionally dissolved in a solvent, in a two-phase mixture of an aqueous alkaline solution, an organic solvent, and a catalyst, preferably an amine compound. The reaction regime can also take place in more than one step. Such processes for producing polycarbonate are in principle known as two-phase interfacial processes, for example from H. Schnell, Chemistry and Physics of Polycarbonates, Polymer Reviews, vol. 9, Interscience Publishers, New York 1964 p. 33 ff. and from Polymer Reviews, vol. 10, “Condensation Polymers by Interfacial and Solution Methods”, Paul W. Morgan, Interscience Publishers, New York 1965, chapter VIII, p. 325, and the essential conditions are therefore familiar to those skilled in the art.

Alternatively, the polycarbonates according to the invention can also be produced by the melt transesterification process. The melt transesterification process is described for example in the Encyclopedia of Polymer Science, vol. 10 (1969), Chemistry and Physics of Polycarbonates, Polymer Reviews, H. Schnell, vol. 9, John Wiley and Sons, Inc. (1964) and in DE-C 10 31 512. In the melt transesterification process, the bisphenols are transesterified in the melt with diaryl carbonates using suitable catalysts and optionally other additives.

Processes for producing polycarbonates using the hydroxy compound of the formula (I) are also known to those skilled in the art. For example, the hydroxy compound of the formula (I) can be used as a chain terminator in an interfacial process for producing polycarbonate in a known manner.

The economic production of the hydroxy compound of the formula (I) as one of its starting materials means that the process of the invention for the production of a polycarbonate is likewise economically advantageous. Corresponding biobased polymers can likewise be produced through the use of biobased compounds.

A further aspect of the present invention provides for the use of at least one Brønsted base for increasing the yield of a hydroxy compound of the formula (I)

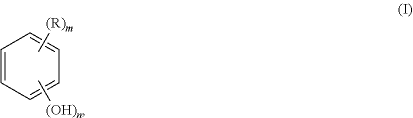

in which

R is a linear or branched alkyl group having 1 to 6 carbon atoms,

n is 1 or 2, and

m is 0, 1, 2, or 3,

in the reaction for the decarboxylation of a carboxylic acid compound of the formula (II) or of a corresponding salt of said carboxylic acid compound of the formula (II)

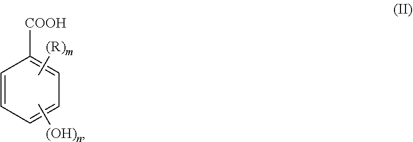

in which R, n, and m are as defined above,

optionally with the use of at least one heterogeneous catalyst.

In this use according to the invention, the preferences and combinations of preferences already described above in relation to the process of the invention apply. In particular, it is preferable that the at least one Brønsted base is selected from the group consisting of sodium hydroxide, potassium hydroxide, sodium phenolate, sodium acetate, sodium phosphate, and any desired mixtures thereof.

### EXAMPLES

### Abbreviations

bara: Absolute pressure in bar

rpm: Revolutions per minute

¹H NMR: Proton resonance spectroscopy

M: Molar concentration in mol/L

aq.: Aqueous solution

Chemicals:

4-Hydroxybenzoic acid (4-HBA): Purity ≥99%, Sigma-Aldrich Chemie GmbH

DM water (H₂O): Demineralized water from the mains supply

Sodium hydroxide (NaOH): Anhydrous, purity ≥97%, Sigma-Aldrich Chemie GmbH

aq. NaOH solution prepared from demineralized water and sodium hydroxide

Phenol: Purity ≥96%, Sigma-Aldrich Chemie GmbH

Hexadeuterodimethyl sulfoxide (DMSO-d6): Purity ≥96%, Euriso-Top GmbH

Zeolite Type Y Catalysts:

Faujasite (product reference: BCR704).

CBV 600 (CAS 1318-02-1), Zeolyst International, Inc., surface area 660 m²/g, pore size 2.43 nm, Si/Al ratio 2.5. The catalyst was calcined prior to use at 300° C. in air for 3 h.

Faujasite, Sigma-Aldrich Chemie GmbH, surface area 567 m²/g, pore size 0.67 nm, Si/Al ratio 1.6. The catalyst was used as received.

General Experimental Procedure:

A 10 mL pressure reactor was charged with 0.5 g of 4-hydroxybenzoic acid, 0.5 mL of solvent (see table 1), and 0.02 g of the respective catalyst (see table 1), flushed with argon as inert gas, and the reactor was closed. The reactor was then pressurized with argon to 3 bara, the mixture was stirred at 800 rpm for 10 min, and the pressure was released to 1.5 bara. This operation was repeated one more time before the reactor was brought to the reaction temperature of 230° C. After the appropriate reaction time (see table 1) at this temperature, the pressure reactor was cooled to room temperature and the pressure released. The reaction mixture obtained was taken up in ethanol, the solid catalyst was separated off by centrifugation (5 min, 5000 rpm, Hettich Universal 320), and the solution was freed of ethanol on a rotary evaporator. The reaction product thus isolated was then investigated by ¹H NMR.

¹H NMR for the Determination of 4-Hydroxybenzoic Acid and Phenol in the Reaction Product:

About 100 mg of the reaction product obtained was dissolved in 0.5 mL of DMSO-d6 and a ¹H NMR spectrum was recorded at 400 MHz on a Bruker Avance 400. The spectra obtained were evaluated on the basis of the specific shifts and integrals shown below.

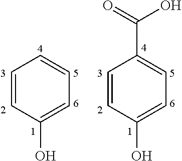

TABLE 1

| | | | Reaction | Reaction product |
|---|---|---|---|---|
| | | | time | [molar ratio from |
| # | Catalyst | Solvent | [h] | ¹H NMR] |
| 1 | Faujasite | H₂O | 2 | 2:1 Phenol/ |
| | | | | 4-hydroxybenzoic acid |
| 2 | Faujasite | aq. NaOH (0.01M) | 2 | Phenol |
| 3 | CBV 600 | H₂O | 2 | 2:1 Phenol/ |
| | | | | 4-hydroxybenzoic acid |
| 4 | CBV 600 | aq. NaOH (0.01M) | 2 | Phenol |
| 5 | — | H₂O | 2 | 1:1 Phenol/ |
| | | | | 4-hydroxybenzoic acid |
| 6 | — | aq. NaOH (0.01M) | 2 | Phenol |
| 7 | — | aq. NaOH (0.01M) | 0.5 | 1:1 Phenol/ |
| | | | | 4-hydroxybenzoic acid |

As can be seen from the table, the addition of a Brønsted base results in a higher yield of phenol under otherwise identical reaction conditions (see Example 1 (comparison) and Example 2 (according to the invention); Example 3 (comparison) and Example 4 (according to the invention) and Example 5 (comparison) and Example 6 (according to the invention)). This applies to different catalysts (Faujasite and CBV 600) and also when a catalyst is not used.

By comparing Examples 4 (comparison), 5 and 6 (according to the invention), it can moreover be seen that the reaction is accelerated by the addition of the Brønsted base. After a reaction time of half an hour with addition of a Brønsted base (Example 7), the yield obtained is the same as after a reaction time of 2 h without addition of a Brønsted base (Example 5).

1. A process for producing a hydroxy compound of the formula (I) 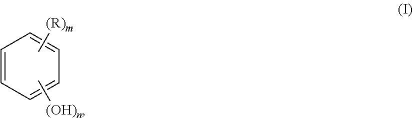
in which
R is a linear or branched alkyl group having 1 to 6 carbon atoms,
n is 1 or 2, and
m is 0, 1, 2, or 3,
by decarboxylation of a carboxylic acid compound of the formula (II) or of a corresponding salt of said carboxylic acid compound of the formula (II) 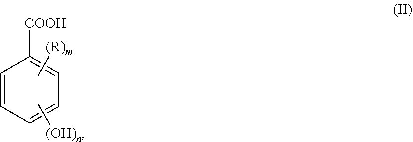
in which R, n, and m are as defined above,
wherein at least one Brønsted base is present during the decarboxylation reaction.

2. The process as claimed in claim 1, wherein the at least one Brønsted base is present in a concentration of 0.0001 mol/L to 20 mol/L based on the entire decarboxylation reaction mixture.

3. The process as claimed in claim 1, wherein the at least one Brønsted base is selected from the group consisting of sodium hydroxide, potassium hydroxide, sodium phenolate, sodium acetate, sodium phosphate, and any desired mixtures thereof.

4. The process as claimed in claim 1, wherein the reaction time of the decarboxylation reaction is longer than 5 minutes and shorter than 48 hours.

5. The process as claimed in claim 1, wherein the decarboxylation reaction is carried out in an aqueous medium.

6. The process as claimed in claim 16, wherein the at least one heterogeneous catalyst used is a zeolite.

7. The process as claimed in claim 6, wherein the zeolite has a faujasite structure.

8. The process as claimed in claim 1, wherein the cation of the salt of the carboxylic acid compound of the formula (II) is selected from the group consisting of alkali metal cations, alkaline earth metal cations, ammonium, phosphonium, cations of manganese, iron, cobalt, nickel, copper, zinc, molybdenum, cadmium, and any desired mixtures thereof.

9. The process as claimed in claim 1, wherein the carboxylic acid compound of the formula (II) or corresponding salt of the carboxylic acid compound of the formula (II) was obtained by fermentation or from sugars, lignocellulose, lignocellulose-containing materials, furans, and/or lignin.

10. The process as claimed in claim 1, wherein the hydroxy compound of the formula (I) is phenol.

11. The process as claimed in claim 1, wherein the carboxylic acid compound of the formula (II) or corresponding salt of the carboxylic acid compound of the formula (II) is selected from the group consisting of 2-hydroxybenzoic acid, 4-hydroxybenzoic acid, and the corresponding salts.

12. A process for producing a diaryl carbonate or a bisphenol, wherein the process comprises the following steps:
(i) executing the process as claimed in claim 1 so as to obtain a hydroxy compound of the formula (I) and
(ii) reacting the hydroxy compound of the formula (I) from step (i) with at least one ketone to obtain a bisphenol or reacting the hydroxy compound of the formula (I) from step (i) with phosgene to obtain a diaryl carbonate.

13. A process for producing a polycarbonate through the polymerization of at least one bisphenol and/or of at least one diaryl carbonate, wherein
the at least one bisphenol and/or the at least one diaryl carbonate was produced by the process as claimed in claim 12.

14. A method comprising increasing the yield of a hydroxy compound of the formula (I) 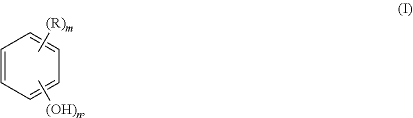
in which
R is a linear or branched alkyl group having 1 to 6 carbon atoms,
n is 1 or 2, and
m is 0, 1, 2, or 3,
in the reaction for the decarboxylation of a carboxylic acid compound of the formula (II) or of a corresponding salt of said carboxylic acid compound of the formula (II) 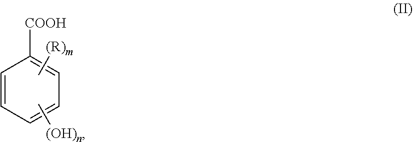
in which R, n, and m are as defined above,
utilizing at least one Brønsted base.

15. The method as claimed in claim 14, wherein the at least one Brønsted base is selected from the group consisting of sodium hydroxide, potassium hydroxide, sodium phenolate, sodium acetate, sodium phosphate, and any desired mixtures thereof.

16. A process as claimed in claim 1, wherein the decarboxylation of a carboxylic acid compound of the formula (II) or of a corresponding salt of said carboxylic acid compound of the formula (II) is with the use of at least one heterogeneous catalyst.

17. A process for producing a polycarbonate through the polymerization of at least one bisphenol and/or of at least one diaryl carbonate, wherein that a hydroxy compound of the formula (I) is produced by the process as claimed in claim 1 and is then used as a chain terminator in the polymerization of the polycarbonate by the interfacial process.

18. A method as claimed in claim 14, wherein the decarboxylation of a carboxylic acid compound of the formula (II) or of a corresponding salt of said carboxylic acid compound of the formula (II) is with the use of at least one heterogeneous catalyst.
